(19) United States
(12) Patent Application Publication
(10) Pub. No.: US 2003/0078416 A1
(43) Pub. Date: Apr. 24, 2003

(54) SUBSTITUTED PHENANTHROLINES

(76) Inventors: YITZHAK TOR, SAN DIEGO, CA (US); DIMITRIOS TZALIS, LA JOLLA, CA (US)

Correspondence Address: FLEHR HOHBACH TEST, ALBRITTON AND HERBERT, FOUR EMBARCADERO CENTER, SUITE 3400, SAN FRANCISCO, CA, 94111

(21) Appl. No.: 08/648,270

(22) Filed: May 15, 1996

Related U.S. Application Data
(60) Provisional application No. 60/016,575, filed on Apr. 30, 1996.

(51) Int. Cl. C07H 19/00; C07H 21/00; C07D 471/04; C08F 30/04; C07H 19/04; C07H 19/06; C07H 19/067; C07H 19/073; C07H 19/09; C08F 130/04

(52) U.S. Cl. 536/28.54; 546/88; 526/241; 536/26.6

(57) ABSTRACT

The invention relates to 1,10 phenanthroline derivatives substituted at the 3-,8-positions.

### FIELD OF THE INVENTION

The invention relates to 1,10-phenanthroline derivatives substituted at the 3-, 8-positions.

### BACKGROUND OF THE INVENTION

Self-assembling supramolecular systems capable of photo-induced electron and energy transfer, and molecular arrays displaying non-linear optical (NLO) properties, exemplify key design targets in materials chemistry. For leading references discussing supramolecular chemistry, see: (a) Lehn, J.-M. Angew. Chem. Int. Ed. Engl. 1990, 29, 1304-1319. (b) Balzani, V.; Scandola, F. Supramolecular Photochemistry Ellis Horwood, N.Y., 1991. (c) Schneider, H.-J.; Dürr, H. (Eds) Frontiers in Supramolecular Organic Chemistry and Photochemistry, VCH, Weinheim, 1991. For a leading reference discussing assemblies with optical non-linearities, see: Marks, T. J.; Ratner, M. A. Angew. Chem. Int. Ed. Engl. 1995, 34, 155-173, and references cited therein. The incorporation of transition metal ions into polymers provides unique opportunities to control the electrical, magnetic and optical properties of the metals. The major approaches taken to date involve incorporating metal ions as side groups attached to the backbone (e.g. polyvinylferrocene), or as part of the polymer main chain (e.g. metallynes). These approaches do not provide full control of the physical properties of the resulting materials and in most cases are not amendable for the synthesis of conducting polymers, as the metal containing polymers are non conjugated.

Ruthenium coordination compounds play a central role in these systems; for example, ruthenium complexes of polypyridine ligands are potential building blocks for luminescent and redox active assemblies as well as for “molecular wires”. For an excellent review of the photophysics and photochemistry of Ru(II) polypyridine complexes, see: Juris, A.; Balzani, V.; Barigelletti, F.; Campagna, S.; Belser, P.; Von Zelewsky, A. Coord. Chem. Rev. 1988, 84, 85-277. For some selected examples for the construction of multinuclear ruthenium complexes, see: (a) Grosshenny, V.; Ziessel, R. J. Organometallic Chem. 1993, 453, C19-C22. (b) Romero, F. M.; Ziessel, R. Tetrahedron Lett. 1994, 35, 9203-9206. (c) Masschelein, A.; Kirsch-De Mesmaeker, A.; Verhoeven, C.; Nasielski-Hinkens, R. Inorg. Chim. Acta 1987, 129, L13-L16. (d) Barigelletti, F.; Flamigni, L.; Balzani, V.; Collin, J.-P.; Sauvage, J.-P.; Sour, A.; Constable, E. C.; Cargill Thompson, A. M. W. J. Am. Chem. Soc., 1994, 116, 7692-7699. (e) Benniston, A. C.; Goulle, V.; Harriman, A.; Lehn, J.-M.; Marczinke, B. J. Phys. Chem. 1994, 98, 7798-7804.

Tuning the electronic properties of the ligands can induce desirable changes in the physical properties of the resulting complexes. In particular, tris(2,2′-bipyridyl)ruthenium(II) exhibits NLO effects; (see Zyss, J. et al., Chem. Phys. Lett. 1993, 206, 409-414; see for a review that summarizes the application of organometallic compounds for non-linear optics Long, N. J. Angew. Chem. Int. Ed. Engl. 1995, 34, 21-38) however, tris([4,4′-dibutylaminostyryl]-2,2′-bipyridyl)-ruthenium(II) shows much larger optical non-linearities (Dhenaut, C. et al., Nature 1995, 374, 339-342).

The rigid framework of 1,10-phenanthroline ligands is an attractive feature for the construction of functional molecular assemblies. Yet, despite their advantageous metal binding properties, 1,10-phenanthroline ligands have rarely been employed for these purposes. (Sammes, P. G. et al., Chem. Soc. Rev. 1994, 23, 327-334; Dietrich-Buchecker, et al., Angew. Chem. Int. Ed. Engl. 1987, 26, 661-663;Chambron, J.-C. et al, J. Chem. Soc., Chem. Comm. 1993, 801-804; Chambron et al., Pure & Appl. Chem. 1995, 67, 233-240; Vögtle, et al., Angew. Chem. Int. Ed. Engl. 1991, 30, 1333-1336; Goodman et al., Tetrahedron Lett. 1994, 35, 8943-8946). This is largely due to the lack of synthetically accessible building blocks. In general, 1,10-phenanthroline ligands substituted at the 2,9 and 4,7 positions are available, while derivatives substituted along the strategic long axis of the molecule, (i.e., at the 3-, 8-positions), have been traditionally difficult to synthesize, requiring low-yield multi-step Skraup reactions sequences which utilize carcinogens like bromoacrolein and produce arsenic rich waste streams; see Case, J. Org. Chem. 16:941-945 (1951). Since the most intense electronic transitions of the phenanthroline ring are polarized along this axis, (Bosnich, B. Acc. Chem. Res. 1969, 2, 266-273) a need existed for the facile synthesis of 1,10-phenanthroline derivatives functionalized at the 3 and/or 8 positions.

Accordingly, it is an object of the invention to provide methods for the bromination of 1,10-phenanthroline at the 3- and/or 8- positions. It is a further object of the invention to provide conjugated derivatives, such as acetylene derivatives, of 1,10-phenanthroline at the 3 and/or 8 position. It is an additional object to provide dendritic derivatives of 1,10-phenanthroline. It is a further object to provide 1,10-phenanthroline covalently attached to nucleic acids via acetylene linkages at the 3 and/or 8 position.

### SUMMARY OF THE INVENTION

In accordance with the objects outlined above, the present invention provides methods for making acetylene derivatives of phenathrolines comprising reacting a 3,8 brominated phenanthroline with an aromatic acetylene.

A further aspect of the invention provides compounds having the formula comprising:

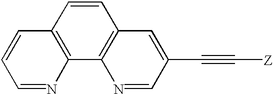

wherein Z is alkyl, substituted alkyl, aromatic or substituted aromatic group.

Additionally provided are compounds having the formula comprising

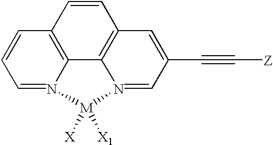

wherein M is a transition metal ion and X and X₁ are co-ligands.

Further provided are polymers having the formula comprising:

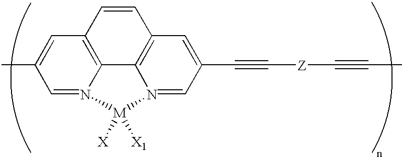

wherein M is a transition metal ion and X and X₁ are co-ligands.

Additionally provided are compounds having the formula:

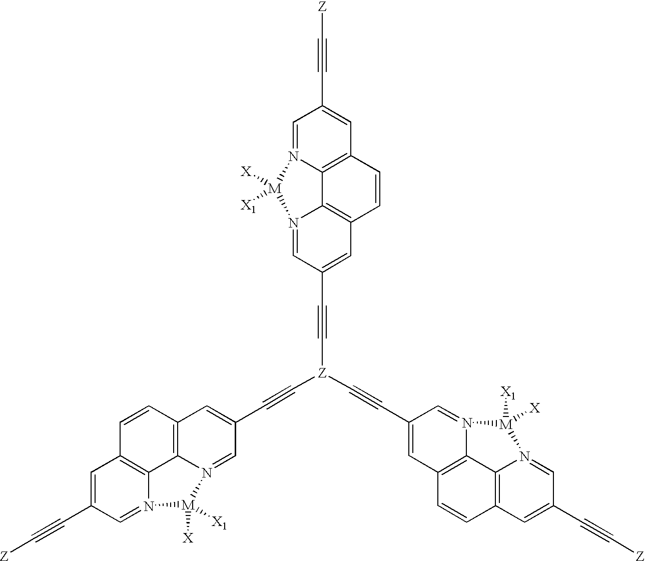

### DETAILED DESCRIPTION OF THE INVENTION

The invention provides compounds comprising derivatives of 1,10-phenanthroline, and methods useful in their synthesis. The 3-,8-positions of 1,10-phenanthroline have special properties. It is very difficult to modify 1,10-phenanthroline at these positions. However, the novel methods disclosed herein allow the facile bromination of 1,10-phenanthroline at one or both of these positions. The brominated 1,10-phenanthroline is then useful in a wide variety of reactions, most particularly in reactions with aromatic and aliphatic acetylenes, acetenes and azo derivatives, to form a wide variety of compounds. In particular, compounds containing the 3- and/or 8-modified 1,10-phenanthroline are used to chelate transition metals. The resulting metal complexes are useful in a wide variety of applications, including novel dendritic materials and for the addition of such transition metal complexes to nucleic acids and other biological compounds.

In one embodiment, the compounds of the invention are modified at at least one of the 3-, 8-positions, and thus have the formula comprising Structure 1:

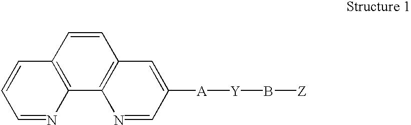

In this embodiment, A and B are each independently either carbon or nitrogen, and Y is a conjugated bond, that is, a bond that contains a sigma (σ) bond and at least one pi (π) bond. Preferred embodiments utilize carbon as both the A and B atoms, thus forming either acetylene (ethynyl; one sigma and two pi bonds; Structure 2) or acetene (ethylene; one sigma and one pi bond; Structure 3), or both nitrogens, thus forming azo bonds (Structure 4), although imine bonds may also be used in some embodiments. Z is an aromatic or alkyl group, as defined below.

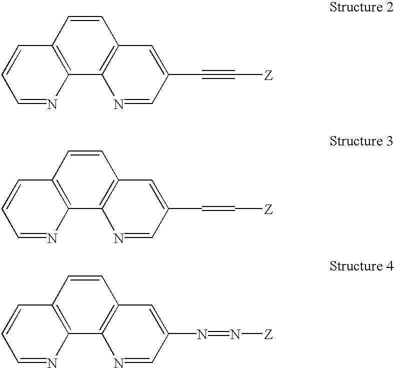

Acetylene linkages are preferred, and the remainder of the disclosure and structures herein will be directed primary to the invention utilizing acetylene linkages. It will be appreciated by those in the art that acetene, azo or imine linkages may be substituted for one or more of the acetylene linkages in any of the structures.

In one embodiment, the compounds of the invention are modified at both the 3- and 8-positions, and thus have the formula depicted in Structure 5:

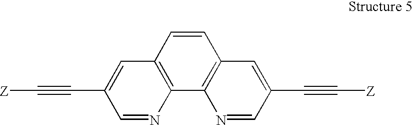

In a preferred embodiment, the compounds of the invention serve as metal chelates, preferably transition metal chelates, and thus the compounds further include a metal ion or atom. That is, the nitrogens of the 1,10-phenanthroline serve as coordination atoms, preferably in conjunction with other ligands, for the chelation of a transition metal atom or ion, as is generally depicted in Structure 6:

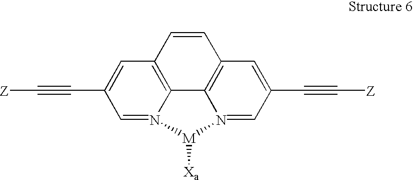

In this embodiment, M is a metal atom, with transition metals being preferred. Suitable transition metals for use in the invention include, but are not limited to, Cadmium (Cd), Copper (Cu), Cobalt (Co), Zinc (Zn), Iron (Fe), Ruthenium (Ru), Rhodium (Rh), Osmium (Os) and Rhenium (Re), with Ruthenium, Rhenium and Osmium being preferred and Ruthenium(II) being particularly preferred.

X is a co-ligand, that provides at least one coordination atom for the chelation of the metal ion. As will be appreciated by those in the art, the number and nature of the co-ligand will depend on the coordination number of the metal ion.

Mono-, di- or polydentate co-ligands may be used. Thus, for example, when the metal has a coordination number of six, two coordination atoms are provided by the nitrogens of the 1,10-phenanthroline, and four coordination atoms are provided by the co-ligands. Thus, n=four, when all the co-ligands are monodentate; n=2, when the co-ligands are bidentate, or n=3, for two monodentate co-ligands and a bidentate co-ligand. Thus generally, n will be from 1 to 10, depending on the coordination number of the metal ion.

In a preferred embodiment, as is generally depicted herein, the metal ion has a coordination number of six and two bidentate co-ligands are used (X and X₁), as is depicted in Structure 7 (corresponding to Structure 2) and Structure 8 (corresponding to Structure 5):

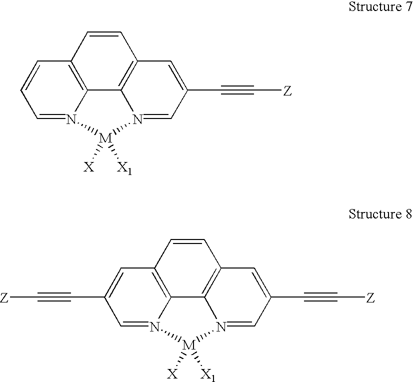

As will be appreciated in the art, the co-ligands can be the same or different. Suitable ligands are well known in the art and include, but are not limited to, NH₂; pyridine; pyrazine; isonicotinamide; imidazole; bipyridine and substituted derivatives of bipyridine; phenanthrolines, particularly 1,10-phenanthroline (abbreviated phen) and substituted derivatives of phenanthrolines such as 4,7-dimethylphenanthroline and the compounds disclosed herein; dipyridophenazine; 1,4,5,8,9, 12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam). In some embodiments, porphyrins and substituted derivatives of the porphyrin family may be used.

Thus, in one embodiment, a single transition metal ion utilizes one, two or three phenathroline derivatives as the ligands.

In the structures depicted herein, Z is an aromatic, substituted aromatic, alkyl or substituted alkyl group or a Silicon (Si) or Tin (Sn) moiety. By “aromatic” or “aromatic group” herein is meant aromatic and polynuclear aromatic rings including aryl groups such as phenyl, benzyl, and naphthyl, naphthalene, anthracene, phenanthroline, heterocyclic aromatic rings such as pyridine, furan, thiophene, pyrrole, indole, pyrimidine and purine, and heterocyclic rings with nitrogen, oxygen, sulfur or phosphorus. Preferred aromatic groups include phenyl groups, pyridine, purine, and pyrimidine groups.

By “substituted aromatic group” herein is meant that the aromatic moiety to which the 1,10-phenanthroline is attached contains further substitution moieties. That is, in addition to the phenanthroline derivative, the aromatic group may be further substituted by any number of substitution moieties. The substitution moiety may be chosen from a wide variety of chemical groups, or biological groups including amino acids, proteins, nucleosides, nucleotides, nucleic acids, carbohydrates, or lipids. That is, any group which contains an aromatic group may serve as the substituted aromatic group. Suitable chemical substitution moieties include, but are not limited to, alkyl, aryl and aromatic groups, amino, nitro, phosphorus and sulfur containing moieties, ethers, esters, and halogens. In some embodiments, as is more fully described below, the substitution moiety of the aromatic group is acetylene linked 1,10-phenanthroline of Structure 2, i.e. two or more 1,10-phenanthrolines share a single Z group, creating multimers and polymers (including dendrimers) of Structure 2.

By “alkyl group” or grammatical equivalents herein is meant a straight or branched chain alkyl group, with straight chain alkyl groups being preferred. If branched, it may be branched at one or more positions, and unless specified, at any position. The alkyl group may range from about 1 to 20 carbon atoms (C1-C20), with a preferred embodiment utilizing from about 1 to about 15 carbon atoms (C1-C15), with about C1 through about C10 being preferred, although in some embodiments the alkyl group may be much larger. Also included within the definition of an alkyl group are cycloalkyl groups such as C5 and C6 rings, and heterocyclic rings with nitrogen, oxygen, sulfur or phosphorus.

By “substituted alkyl group” herein is meant an alkyl group further comprising one or more substitution moieties, as defined above.

By “silicon moiety” herein is meant an alkylsilyl group, with trialkylsilyl being preferred and trimethylsilyl (TMS) being particularly preferred.

By “tin moiety” herein is meant an alkylstannyl group.

In a preferred embodiment, the phenanthroline is linked to an aromatic or alkyl group containing a substitution moiety such that the phenanthroline is conjugated with the substitution moiety. In the case of a substituted alkyl or substituted aromatic containing an alkyl moiety, this may require that the alkyl group itself be unsaturated so as to facilitate conjugation.

In a preferred embodiment, for example when the compounds of the invention include a transition metal ion, the Z group comprises a biological moiety such as a nucleotide or a nucleic acid. In such an embodiment, the preferred attachment is through the nucleoside base; i.e. an acetylene group is attached to the base for example as depicted below in Structure 9. That is, the aromatic heterocyclic base is an aromatic group, and the remainder of the nucleotide or nucleic acid comprises the substitution moiety of the aromatic group. By “nucleoside” herein is meant a purine or pyrimidine nitrogen base bonded to a carbohydrate such as a ribose, i.e. adenosine, guanosine, thymidine, cytidine, and uridine. By “nucleotide” herein is meant a nucleoside further containing a phosphate group. Specifically included within the definition of nucleotide is the phosphoramidite form of a nucleotide, as is depicted in Structure 11. By “nucleic acid” herein is meant at least two nucleotides covalently linked together. A nucleic acid of the present invention will generally contain phosphodiester bonds, although in some cases, as outlined below, a nucleic acid may have an analogous backbone, comprising, for example, phosphoramide (Beaucage et al., Tetrahedron 49(10):1925 (1993) and references therein; Letsinger, J. Org. Chem. 35:3800 (1970)), phosphorothioate, phosphorodithioate, O-methylphophoroamidite linkages (see Eckstein, Oligonucleotides and Analogues: A Practical Approach, Oxford University Press), or peptide nucleic acid linkages (see Egholm, J. Am. Chem. Soc. 114:1895 (1992); Meier et al., Chem. Int. Ed. Engl. 31:1008 (1992); Nielsen, Nature, 365:566 (1993)). The nucleic acids may be single stranded or double stranded, as specified. The nucleic acid may be DNA, RNA or a hybrid, where the nucleic acid contains any combination of deoxyribo- and ribo-nucleotides, and any combination of uracil, adenine, thymine, cytosine and guanine. Included within the definition of nucleic acid are single nucleosides and nucleotides, and the phosphoramidite form of nucleotides, as is described herein.

Structures 9, 10, and 11 depict a 3-acetylene-phenanthroline modified uridine nucleoside, nucleotide, and phosphoramidite nucleotide respectively, and Structure 12 depicts a uridine attached to a peptide nucleic acid backbone subunit, all attached to the 1,10-phenanthroline via the acetylene linkage described herein, in the absence of metal ions and co-ligands. Structures 9, 10, 11 and 12 depict the attachment via the 5 position of the uracil base, although attachment at the 6 position are also possible. R can be either H (deoxyribose) or OH (ribose).

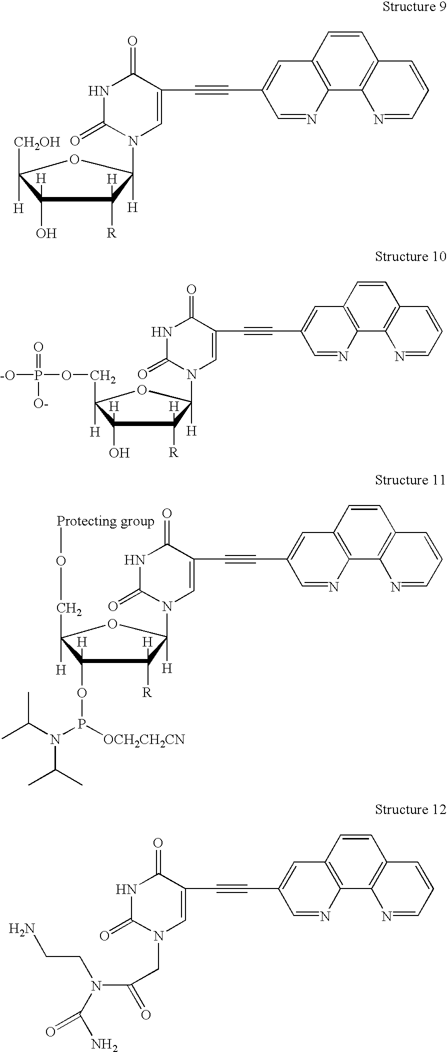

These structures may also include the transition metal ion and co-ligands, as will be appreciated in the art. The protecting group depicted in Structure 8 may be any number of known protecting groups, such as dimethoxytrityl (D)MT); see generally Greene, Protecting Groups in Organic Synthesis, J. Wiley & Sons, 1991.

Similarly, linkages such as acetylene linkages may also be made to the bases of the four other nucleic acids, cytosine, thymine, adenine, and guanine. For cytosine, the linkage is preferably via the 5 or 6 positions. For thymine, the linkage is preferably via the 5 and 6 positions. For adenine, the linkage is preferably via the 8 position. For guanine, the linkage is preferably via the 8 position.

As will be appreciated by those in the art, the phenanthroline compounds of the present invention may also be attached to amino acids and proteins. Thus, for example, covalent attachment may be done through the amino acid side chains.

In a preferred embodiment, the Z group contains one or more acetylene-linked 1,10-phenanthrolines as the substitution group. Thus, as will be appreciated by those in the art, multimers and polymers or dendrimers of the basic compound of Structure 1 can be made. By “multimers” herein is meant two or more 1,10-phenanthrolines linked via a single Z group. That is, a single Z group has two or more phenanthroline groups attached. For example, the Z group may be substituted by one or more acetylene-linked 1,10-phenanthrolines, as is depicted in Structure 13 (in the absence of a transition metal) or Structure 14 (in the presence of metal ions) for two 1,10-phenanthrolines, or Structure 15 (in the presence of metal ions) for three 1,10-phenanthrolines. Structure 15 utilizes phenyl as an aromatic Z group, but as will be appreciated in the art, other Z groups may be utilized.

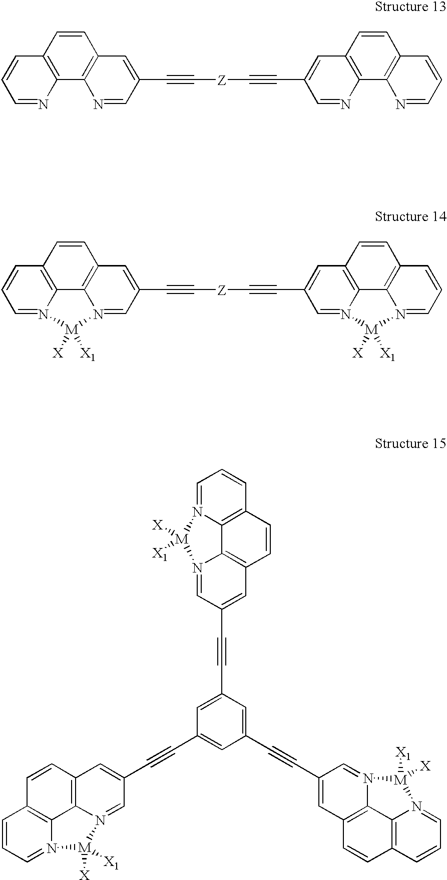

When the multimers are further extended, that is, the 1,10-phenanthroline is substituted, for example to form acetylene linkages at both the 3- and the 8-position, polymers may be formed. The polymers of the invention have the general structure shown below, depicted below with the metal ion and co-ligands:

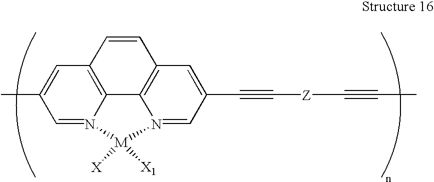

As will be appreciated by those in the art, n can range from quite small, such as n=2, to very large, from greater than about 100, 1,000, 10,000 or 100,000 or more.

In this embodiment, various metal ions and Z groups (substituted or unsubstituted) may be used. That is, the polymer may comprise more than one type of metal ion and more than one type of Z group. In addition, as outlined below, the 1,10-phenanthroline may be additionally substituted, and thus substituted and unsubstituted 1,10-phenanthroline may be used. In a preferred embodiment, substitution positions are chosen for linear molecules, such that the molecules are fully conjugated. Alternatively, such as depicted in Structures 15 and 17, the molecules are non-linear. In this embodiment, Z groups may be used that contain three or more acetylene-linked 1,10-phenanthroline groups, thus forming “cross-linking” structures, or dendrimers.

Thus, in a preferred embodiment, the 1,10-phenanthrolines depicted in Structure 15 have additional Z groups at the 8-position, as is depicted below in Structure 17 (in the presence of metal ion and co-ligands):

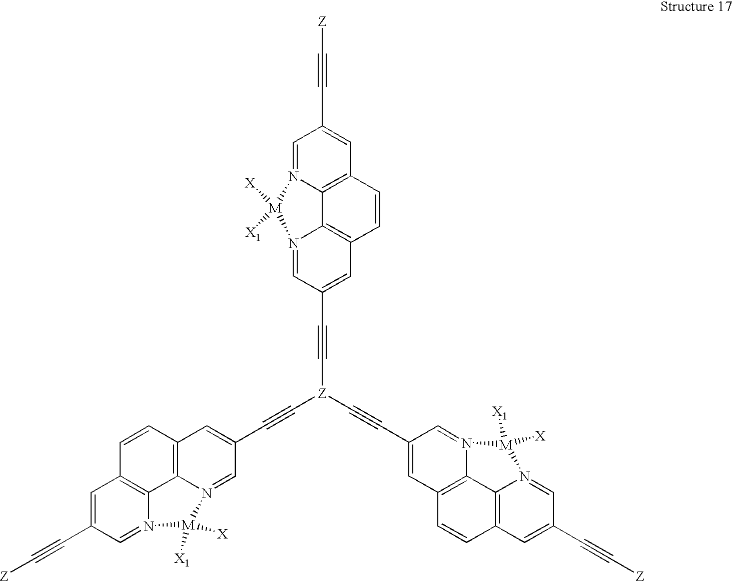

In this embodiment, the Z groups are preferably aromatic groups, with phenyl being preferred.

In addition, the 1,10-phenanthroline may be substituted at other positions in addition to the 3-,8-position, as defined above, as depicted in Structure 18 in the absence of the metal ion and co-ligands. R may be a wide variety of R substitution groups, as defined above. In some embodiments, adjacent R groups form cyclic, preferably aromatic groups, conjugated to the phenanthroline. If the R groups are added prior to bromination, the R groups preferably do not interfere with the bromination at the 3 and/or 8 positions.

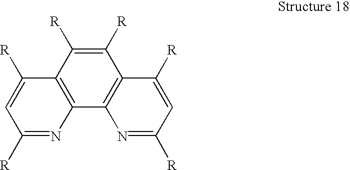

As will be appreciated by those in the art, the compounds of the invention generally are charged, due to the metal ion.

The invention further provides methods for the synthesis of the compounds depicted herein.

The invention provides methods for the bromination of 1,10-phenanthroline at the 3 and/or 8 positions. The method comprises reacting an acid salt of 1,10-phenanthroline with bromine in the presence of a solvent such as nitrobenzene, bromobenzene, or chlorobenzene. By “acid salt” herein is meant a compound derived from the acids and bases in which only a part of the hydrogen of the acid is replaced by a basic radical. Preferred acid salts include the monohydrochloride monohydrate of 1,10-phenanthroline (1 in Scheme I). In some embodiments, the acid salt form is generated in situ and thus is not required as a starting material. The solvent used may be nitrobenzene, bromobenzene, or chlorobenzene. The method is schematically depicted in Scheme I:

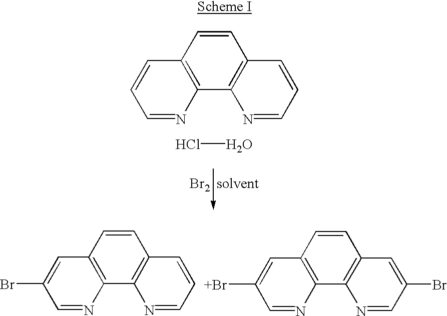

Scheme I generally results in a mixture of 3-bromo-phenanthroline and 3,8-bromo-phenanthroline, which are easily separated using a variety of techniques in the art, such as silica gel purification and flash column chromatography.

The 3- or 3,8 brominated 1,10-phenanthroline is then used in a variety of reactions to form the compounds of the invention.

In a preferred embodiment, palladium-mediated cross coupling as is known in the art is used to react the brominated 1,10-phenanthroline with a Z group such as an aromatic acetylene to form the compounds of the invention, as is generally depicted in Scheme II. Alternatively, the brominated 1,10-phenanthroline is reacted with an acetylene, to form a 3- or 3,8-acetylene-phenanthroline, which then may be reacted with a halogenated aromatic Z group to form the compounds, as is depicted in Scheme III.

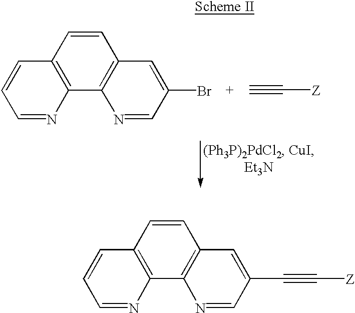

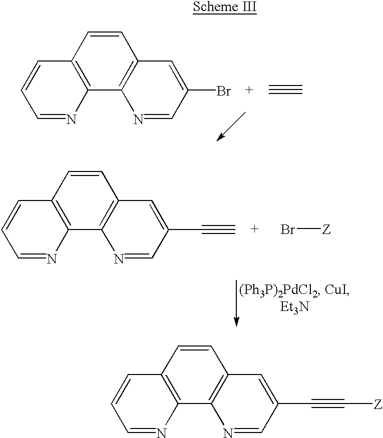

Scheme II and III are depicted with a single bromine on the 1,10-phenanthroline. The use of the doubly brominated 1,10-phenanthroline permits the incorporation of two Z groups at the 3- and 8-positions coupled by acetylene linkages. As is discussed below, the polymers of the invention can be generated using such 3,8-bifunctional phenanthrolines.

Suitable palladium-mediated cross coupling conditions are well known in the art. See for example, K. Sonogashira et al., Tetrahedron Lett. 1975, 4467; L. S. Hegedus, in Transition Metals in the Synthesis of Complex Organic Molecules, University Science Books, Mill Valley, Calif. 1994; pp. 65-127; R. Rossi et al., Org. Prep. Proc. Int. 1995, 27, 127; K. C. Nicolaou et al., Chem. Eur. J. 1995, 1, 318; M. D. Shair et al., J. Org. Chem. 1994, 59, 3755; Z. Xu et al., J. Am. Chem. Soc., 1994, 116, 4537; D. L. Pearson et al., Macromolecules, 1994, 27, 2348; DiMagno et al., J. Org. Chem. Soc. 58:5983 (1993); S. Prathapan et al., J. Am. Chem. Soc. 1993, 115, 7519; R. W. Wagner et al, J. Org. Chem. 1995, 60, 5266; J. Seth etal. J. Am. Chem. Soc. 1994, 116, 10578; V. S.-Y Lin et al., Science 1994, 264, 1105; and H. L. Anderson et al., Angew. Chem. Int. Ed. Engl. 1990, 29, 1400; all of which are hereby expressly incorporated by reference. Hydrogenation can result in the acetene derivatives.

Once the compounds are generated, transition metal ions and co-ligands can then be added, using techniques well known in the art.

In a preferred embodiment, the palladium-mediated cross coupling reaction is done with the compounds already containing the transition metal ions and co-ligands. Without being bound by theory, it appears that the electron withdrawing properties of the transition metal ion facilitates the addition reaction, allowing a simple single step synthesis, as is depicted in Scheme IV (3-brominated 1,10-phenanthroline and aromatic acetylene) and Scheme V (3-acetylene-phenanthroline and aromatic bromine):

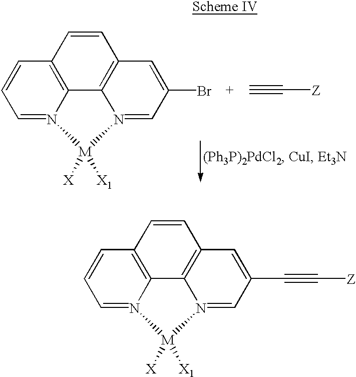

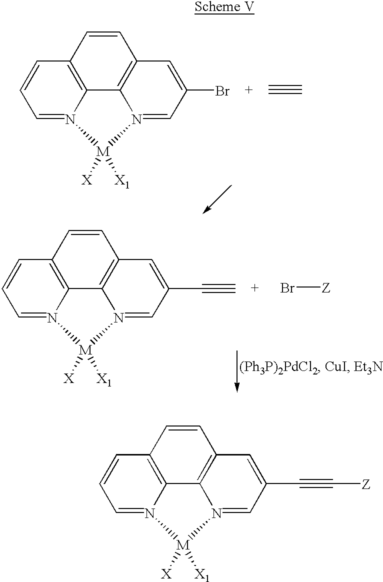

The aromatic acetylenes may be made using techniques well known in the art. See for example, Nguyen et al., Synlett 1994, 299-301, expressly incorporated herein by reference. Many aromatic acetylenes are commercially available, such as phenylacetylene, 4-ethynyltoluene, or are easily generated from brominated precursors; for example, 1,3,5 tribromobenzene is commercially available.

In a preferred embodiment, the compounds of the invention are attached to nucleosides, nucleotides, and nucleic acids. Generally, halogenated nucleosides are commercially available. For example, uridine iodinated at the 5-position may be used in either Scheme III or Scheme V. Similarly, the phosphoramidite derivative of the nucleotides may be made as is known in the art.

Thus, in a preferred embodiment, the invention further provides methods of generating nucleic acids comprising the compounds of the invention. The method comprises incorporating a phosphoramidite nucleotide containing the acetylene-linked 1,10-phenanthroline into a synthetic nucleic acid.

As outlined herein, a preferred embodiment utilizes polymers or dendrimers of the compounds of the invention. Polymers can be generated by using 3,8 halogenated 1,10-phenanthroline, and any number of Z groups.

In a preferred embodiment, the polymers are generated using a single type of Z group, preferably an aromatic group. A preferred embodiment utilizes 1,3,5-triethynylbenzene as an aromatic acetylene. Alternative embodiments utilize other Z groups.

In an alternate embodiment, the polymers are generated using more than one type of Z group, thus forming co-polymers. As will be appreciated by those in the art, any number of different Z groups may be used.

The compounds of the invention are purified if necessary, using techniques known in the art.

Once made, the compounds of the invention find use in a number of applications. The phenanthroline compounds of the invention are fluorescent, and in a preferred embodiment, may be used as labels. Thus, for example, nucleic acid probes may be made and labelled with the compounds of the invention, for the detection of target sequences, for example for diagnostic purposes.

In an additional embodiment, the compounds are used to attach metal ions to biological moieties such as nucleic acids and proteins for energy and electron transfer purposes.

In a preferred embodiment, the compounds of the invention are used to make multimetallic assemblies for the study of energy and electron transfer, and find application in the area of non-linear optics, liquid crystals, electrochromic display devices, photonic and electrochemical sensing devices, energy conversion systems, information recording and “molecular wires”.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### Example 1

### Bromination of 1,10-phenanthroline

Method A: Nitrobenzene as Solvent

Phenanthroline substituted in either the 3 or the 3 and 8 positions have been traditionally difficult to functionalize, requiring low-yield multi-step Skraup reaction sequences (see Case, supra). Conventional wisdom advises that simple bromination of 1,10-phenanthroline is poor and unselective. See Katritzky et al., Electrophilic Substitution of Heterocycles: Quantitative Aspects (Vol. 47 of Adv. Heterocycl. Chem.); Academic Press: San Diego, 1990; Graham, in the Chemistry of Heterocyclic Compounds; Allen, Ed. Interscience Publishers, Inc.

New York 1958, pp386-456. A direct bromination reaction gives low yields of di-, tri- and tetrabrominated 1,10-phenanthroline and traces of the 3- and 5-bromo derivatives has been reported; see Denes et al., J. Prukd. Chem. 320:172-175 (1978).

However, starting with the commercially available 1,10-phenanthroline monohydrochloride monohydrate, the reaction with bromine using nitrobenzene as the solvent gives 3-bromo-phenanthroline and 3,8-bromo-phenanthroline as major products. In a typical procedure, a solution of the 1,10-phenanthroline monohydrochloride monohydrate(10 g, 43 mmol) in nitrobenzene (20 ml) was heated to 130-140° C. in a 250 ml 3-neck flask. Bromine (3.31, 64 mmol in 9.3 ml nitrobenzene) was added dropwise over a period of 1 hr. Upon the addition of bromine, the 1,10-phenanthroline went into solution. After stirring for 3 hr at the same temperature, the reaction mixture was cooled to room temperature, treated with concentrated ammonium hydroxide (100 ml) and extracted with dichloromethane (3×50 ml). The combined organic layers were washed with water (3×50 ml) and dried (MgSO₄). Concentration in vacuum afforded a suspension of the products in nitrobenzene. The nitrobenzene was removed by dissolving the suspension in dichloromethane (10 ml) and filtering it through silica gel (300 ml) using dichloromethane as the eluent. After the nitrobenzene eluted out, the products were recovered by gradually increasing the polarity of the eluent up to 10% MeOH in CH₂Cl₂. Flash column chromatography (0.6% MeOH in CH₂Cl₂) afforded 3-bromo-phenanthroline (3.6 g, 33% yield, m.p. 164-167° C.) and the 3,8-bromo-phenanthroline (2.4 g, 17% yield, m.p. 270-273° C.) as white powders. Higher solvent polarity (10% MeOH in CH₂Cl₂) elutes unreacted 1,10-phenanthroline (ca. 4 g) that can be recycled.

Variations of the amount of bromine, reaction time, or temperature influence the outcome of the reaction. Attempts to push the reaction to completion usually resulted in higher yields of the 3,8-bromo-phenanthroline but at the same time led to the generation of various other brominated derivatives. Under the present conditions, ca. 90% of crude 1,10-phenanthroline containing products can be accounted for as unsubstituted 1,10-phenanthroline, the 3-bromo product and the 3,8-bromo product. The remaining 10% contains several other brominated by-products (5-bromo-, 3,5,8-tribromo- and 3,5,6,8-tetrabromo-phenanthroline) that can be removed by column chromatography.

Method B: Bromobenzene as Solvent

3.01 g, 1 eq, 0.128 moles of 1,10-phenanthroline was placed in a round bottom flask (2 neck) with a stir bar. 200 ml of bromobenzene was added and the mixture was sonicated for 20 minutes. After heating the mixture to 135° C. with reflux and stirring, 1 drop of bromine mixture (2 ml of bromine and 100 mls of bromobenzene) was added per minute. The reaction was monitored by TLC (aluminum oxide, 3% methanol/methylene chloride). Reflux was continued for 30 minutes after addition was complete. The reaction was then quenched with aqeuous ammonia with sonication for 20 minutes. The aqueous phase was removed, and washed with methylene chloride (X2). The organic phase was washed three times with saturated NaCl solution, and the organic phases combined, and dried with anhydrous magnesium sulfate, filtered, and the bromobenzene evaporated under reduced pressure. The separation of the two forms was done by flash chromatography (silica gel, 0.3% methanol/methylene chloride under dibromophenanthroline eluted, 1% methanol/methylene chloride until monobromophenanthroline eluted. The solvent was evaporated under reduced pressure.

### Example 2

### Synthesis of 3,8-bis(aromaticethynyl)-phenanthroline in the absence of transition metal ions

The following scheme was used:

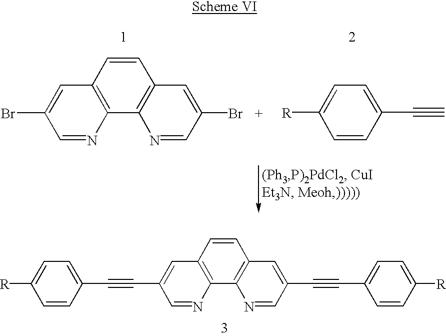

The new ligands are synthesized by cross-coupling reactions between 3,8-dibromo-1,10-phenanthroline (1) as described in Example 1 and substituted phenylacetylenes (2) in the presence of (Ph₃P)₂PdCl₂ and CuI under sonication at room temperature (Scheme). In a typical reaction, a degassed solution of phenylacetylene (0.26 ml, 2.5 mmol) in triethylamine (8 ml) and methanol (4 ml) was added under argon to a reaction flask containing 1 (0.1 g, 0.3 mmol), (Ph₃P)₂PdCl₂ (16 mg, 0.03 mmol) and CuI (10 mg, 0.05 mmol). The mixture was sonicated at room temperature under argon for 2-4 hr. The reaction mixture was dissolved in dichloromethane (50 ml), washed with aqueous KCN and water. Drying (MgSO₄) and evaporation afforded the crude product. Flash chromatography (1% methanol/dichloromethane) followed by recrystallization from chloroform afforded 3a. ¹H NMR (CDCl₃) d 9.31 (d, J=1.9 Hz, 2H, H2,9), 8.41 (d, J=1.9 Hz, 2H, H4,7), 7.83 (s, 2H, H5,6), 7.65 (m, 4H, phenyl-H2), 7.36 (m, 6H, phenyl-H3,4); ¹³C NMR (CDCl₃) d 152.4, 144.3, 138.0, 131.9, 129.0, 128.5, 128.0, 126.8, 122.3, 119.8, 94.0, 86.3. Reactions performed at room temperature without sonication proceed much slower. The effect of sonication was not thoroughly investigated, although it is possible that sonication promotes the reaction by facilitating the solubilization of 1 in the reaction medium. Although reactions at elevated temperatures yielded the desired products, they were accompanied by the formation of undesired by products. Table I summarizes selected data for the new ligands.

TABLE 1
Preparation and selected spectral data for 3.

| Ligand | R | Yield^{a} | MS^{b} | UV^{c} |
|---|---|---|---|---|
| 1,10-ph^{d} | — | — | — | 230(5.1),264(3.0),280(1.2) |
| 3a | H | 90% | 380.1302(380.1313) | 284(5.3),340(5.7),354(4.1) |
| 3b | CH₃ | 87% | 408.1607(408.1626) | 286(4.2),346(5.1),360(4.6) |
| 3c | OCH₃ | 89% | 440.1516(440.1524) | 290(4.0),^{e} 352(5.5),368(4.9) |
| 3d | CF₃ | 43% | 516.1060(516.1061) | 286(5.4),338(6.4),352(5.5) |

^{a}Isolated yields of chromatographically pure products based on 1.
^{b}Observed and (in parenthesis) calculated EI high resolution mass spectrum.
^{c}UV spectra of 1 × 10⁻⁵ M solutions in acetonitrile. The absorption maxima are given in nm and 10⁻⁴ε (in parenthesis) is given in Mhu −1cm⁻¹. Prominent shoulders are italicized.
^{d}The data for 1,10-phenanthroline is given for comparison. ^{e}A broad absorption between 268-290 nm is observed.

Comparing the ultraviolet spectra of the new ligands 3 to that of the parent 1,10-phenanthroline shows a substantial red-shift of the p-p* transitions and a change in the relative intensity of the two major bands (Table 1). The higher energy transition in 3a is shifted by 54 nm compared to that of phenanthroline, while the lower energy transition is shifted by 76 nm. The two major bands in the UV spectrum of phenanthroline have been assigned to the long-axis polarized β (230 nm) and β′ (264 nm) transitions; see: Bray, R. G. et al., Aust. J Chem. 1969, 22, 2091-2103. The major transitions of the new ligands are only tentatively assigned here. A careful study of the absorption and fluorescence spectra of the conjugated ligands under various conditions is required for a full analysis. Similar effects have been observed in other phenylacetylene conjugated aromatic systems; for example, the major absorption band of 9,10-bis(phenylethynyl)-anthracene is red-shifted by 73 nm compared to anthracene. See Maulding et al., J. Org. Chem. 34:134-136 (1969). This is indicative of a substantial electron delocalization through the ethynyl groups. The lower energy absorption maximum of the methoxyphenyl derivative 3c is 6 nm red-shifted compared to the toluyl derivative 3b which is red-shifted by 6 nm compared to the phenyl derivative 3a. Clearly, the absorption maxima are affected by the remote ring substituents which support an extended conjugation.

In a typical reaction, the ligand 3a (0.1 g, 0.26 mmol) in degassed DMF (10 ml) was treated under argon with a solution of K₂RuCl₅ (33 mg, 0.08 mmol) in water (4 ml) containing 1 drop of 6N HCl. The solution was refluxed for 1 h. Sodium hypophosphite (38 mg, 0.44 mmol) in water (1 ml) was added, and reflux was continued for 1 h. After cooling to 600° C., the reaction mixture was treated with potassium hexafluorophosphate (48 mg, 0.26 mmol) as a 10% aqueous solution, cooled to RT and concentrated in vacuo. Silica-gel chromatography using 1% aqueous 0.5 M KNO₃ in acetonitrile as eluent afforded Ru(3a)₃. ¹H NMR (CD₃CN) d 8.75 (d, J=1.3 Hz, 2H, H2,9), 8.27 (s, 2H, H5,6), 8.18 (d, J=1.3 Hz, 2H, H4,7), 7.45 (m, 10H, phenyl).

Upon complex formation, the electronic transitions of 1,10-phenanthroline remain largely unmodified except for a small hypsochromic effect of the two major transitions (Table 2). In contrast, the Ru(II) complexes of ligands 3 show a different behavior (Table 2). Although the higher energy transitions around 280 nm are blue-shifted upon Ru(II) complexation, the lower energy transitions at ca. 340 nm are red-shifted (compare Tables 1 and 2). The latter seem to be more sensitive to the nature of the substituent on the phenyl rings with the methoxy derivative Ru(3c)₃ suffering the largest shift of more than 25 nm. The visible metal to ligand charge transfer (MLCT) bands, while red-shifted by ca. 30 nm in Ru(3)₃ compared to Ru(1,10-phen)₃, appear at the same wavelength for all derivatives. MCLT bands in Ru(II) complexes of other substituted phenanthrolines have been shown not to be very sensitive to the nature of the substituents. See for example Lin etal., J. Am. Chem. Soc. 98:6536-6544 (1976).

TABLE 2
Preparation and selected spectral data for Ru(II) complexes of ligands 3.

| Complex | R | Yield^{a} | MS^{b} | UV-vis^{c} |
|---|---|---|---|---|
| Ru(1,10-Ph)₃ | — | 77% | — | 224(7.2), 262(9.6), 290(2.0), |
| | | | | 446(1.6) |
| Ru(3a)₃ | H | 60% | 1242(M⁺) | 280(6.3),294(5.8), 356(6.5), |
| | | | | 376(5.0), 474(0.72) |
| Ru(3b)₃ | CH₃ | 86% | 1325(M²⁼ − H⁺) | 276(8.7), 296(7.4), 364(9.8), |
| | | | 1471(M²⁼ +PF₆) | 382(8.2), 474(0.97) |
| RU(3c)₃ | OCH₃ | 94% | 1442(M⁺) | 274(9.3), 300(5.7), 378(8.2), |
| | | | | 394(7.7), 472(0.97) |

^{a}Isolated yields of chromatographically pure complexes (based on 3) as their PF₆-salts.
^{b}Positive FAB mass spectrum.
^{c}UV-vis spectra were taken in acetonitrile. Absorption maxima are given in nm and 10⁻⁴ε (in parenthesis) is given in M⁻¹cm^{-1. The major bands are bolded and prominent shoulders are italicized.}

### Example 3

### Synthesis of 3,8-bis(aromaticethynyl)-phenanthroline in the presence of transition metal ions

The complex [(bpy)₂Ru(3-bromo-1,10-phenanthroline)]²⁺(PF₆-)₂ (1) is an attractive building block for the synthesis of multimetallic Ru(II) arrays using cross-coupling methodology. The 1,10-phenanthroline ligand is substituted at the 3-position which is sterically and geometrically favored and provides electronic conjugation. Tzalis et al., Tetrahedron Lett. 36:6017 (1995). The Ru(II) complexed 3 -bromo-1,10-phenanthroline is expected to be relatively electron-deficient and to therefore undergo facile oxidative-addition reactions. Furthermore, the phenanthroline's nitrogens are “masked”, and complications due to complexation of the transition-metal catalysts are prevented. Suffert et al., Tetrahedron Lett. 32:757 (1991).

Treating a DMF solution of 4 (shown below) with 4-ethynyltoluene at room temperature for 1 hour in the presence of (Ph₃P)₂PdCl₂, CuI and Et₃N proceeds smoothly to afford 6 in excellent yield (Table 3).

TABLE 3
Preparation and selected spectral data for Ru^{II} complexes.

| Complex | Yield(%)^{a} | MS^{b} | UV-VIS^{c} |
|---|---|---|---|
| 4 | 68 | 819.3(M⁺) | 236(4.2), 272(6.6), |
| | | | 286(6.5), 448(1.5) |
| 5 | 68 | 763(M⁺) | 238(4.2), 276(6.3), |
| | | | 286(6.1), 450(1.2) |
| 6 | 91 | 853.5(M⁺) | 244(4.2), 286(7.6), |
| | | | 346(3.0), 452(1.3) |
| 7 | 86 | 327(M⁴⁺) | 238(8.2), 286(15.3), |
| | | | 362(8.1), 440(2.9) |
| 8 | 73 | 347(M⁴⁺) | 238(7.6), 286(13.8) |
| | | | 368(8.4), 440(2.6) |
| 9 | 70 | 554(M⁴⁺) | 244(14.4), 286(24.5), |
| | | | 338(11.8), 440(3.6) |

^{a}Isolated yields of recrystallized complexes as their PF₆-salts. The yields reported for complexes 6 through 9 represent the reaction yields of 1 with the corresponding acetylene (see text).
^{b}Electrospray Ionization Mass Spectrum. The observed peaks correspond to [M-nPF₆-]ⁿ⁻
^{c}UV-VIS spectra were taken in acetonitrile. Absorption maxima are given in nm and 10⁻⁴ε (in parenthesis) is given in dm³ mol⁻¹ cm⁻¹.

A presentative procedure for the palladium-mediated cross-coupling reactions between 4 and aromatic acetylenes is as follows. A mixture of 4 (50 mg, 0.052 mmol), (Ph₃P)₂PdCl₂ (4 mg, 0.0057 mmol) and CuI (0.5 mg, 0.0026 mmol) was treated with a degassed solution of 4-ethynyltoluene (11 μl, 0.11 mmol) in DMF (5 ml) and triethylamine (3 ml) for 1 hour at room temperature under Argon. The crude reaction mixture was evaporated to dryness and the product 6 was obtained in 91% yield as an orange-red powder after successive crystallizations from dichloromethane-ethanol. Selected data for 3: ¹H NMR (500 MHz, CD₃CN) d 8.72 (d, 1H, H2_{Phen}), 8.62 (d, 1H, H9_{Phen}), 8.56-8.49 (m, 4H, H_{d bpy}), 8.27 (d, 1H, H5_{Phen}), 8.22 (d, 1H, H6_{Phen}), 8.19 (d, 1H, H4_{Phen}), 8.12-8.07 (m, 3H, H7_{Phen}, 2H_{g bpy}), 8.04-7.99 (m, 2H, H_{g bpy}), 7.86 (d, 1H, H_{a bpy}), 7.81 (d, 1H, H_{a bpy}), 7.74 (dd, 1H, H8_{Phen}), 7.65 (d, 1H, H_{a bpy}), 7.52 (d, 1H, H_{a bpy}), 7.48-7.45 (m, 4H, 2H_{b bpy}, 2Hₚₕₑₙₗ), 7.28-7.23 (m, 4H, H_{b bpy}, 2Hₚₕₑₙₗ), 2.24 (s, 3H, CH₃). IR (film, NaCl)nₘₐₓ 2215 cm⁻¹ (C∫C).

All Ru(II) complexes were synthesized as their PF₆-salts and showed spectroscopic data (UV-VIS, IR, NMR and MS) consistent with the assigned structure. Electrospray Ionization Mass Spectrometry has been found particularly useful in analyzing these complexes due to the characteristic formation of multiply charged species with typical isotopic distribution. Note that the binuclear and trinuclear complexes are formed as a mixture of stereoisomers. No attempt has been made to resolve these complexes at this point.

Similarly, reacting 4 with 1,4-diethynylbenzene, or 4,4′-diethynyl-1,1′-biphenyl affords the bimetallic complexes 7 and 8, respectively, in good yields. The same mild reaction conditions are applied for the coupling of 1,3,5-triethynylbenzene with 3 equivalents of 4 to afford the trinuclear complex 9 in 70% yield. Cross-coupling reactions of the Ru(II) complex containing the alkyne functionality with aromatic electrophiles have been found to proceed efficiently as well. Thus, treating [(bpy)₂Ru(3-ethynyl-1,10 -phenanthroline)]²⁺(PF₆-)₂ (5) with 1,4-diiodobenzene or 4,4′-diiodobiphenyl under the same reaction conditions, affords the binuclear Ru(II) complexes 7, and 8, in 43% and 56% yield, respectively. In general, the reactions of 5 with aromatic iodides proceed slower than the reactions of 4 with aromatic acetylenes.

The compounds synthesized represent a novel family of multi Ru(II) complexes of various structures and spectral properties (Table 3). The parent complex 4 exhibits two main absorption bands at 272 and 286 nm due to the overlapping π-π* transitions of the bpy and phenanthroline ligands. Although the major band of the bpy appears to remain largely unchanged, extending the conjugation of the phenanthroline ligand results in the appearance of a lower energy band above 330 nm (Table 3). For example, in addition to a strong absorption at 286 nm, 6 shows a new band at 346 nm. This lower energy π-π* transition is further red-shifted with increasing conjugation as is evidenced when comparing the spectrum of 6 to that of 7 (362 nm). The binuclear complex 8 shows similar behavior to that of 7, indicating a substantial electronic conjugation between the two phenanthroline ligands through the biphenyl ring. In contrast, the lower energy π-π* transition of the phenanthroline ring in the trinuclear complex 9 appears at a much shorter wavelength (338 nm) as compared to 7 (362 nm) and 8 (368 nm), and is almost overlapping with that of the mononuclear complex 6 (346 nm). This indicates that each of the metal centers in 9 is electronically isolated and is not involved in π-conjugation. The visible metal to ligand charge transfer (MLCT) bands appear around 440 nm for all derivatives. This somewhat unexpected behavior has been observed in other mono- and binuclear Ru(II) complexes (see Bolger et al., J. Chem. Soc. Chem. Commun. 1799 (1995) and Tzalis et al., supra). Nevertheless, the different nuclearity of the complexes 6, 7 (8) and 9 is beautifully evident from the approximate 1:2:3 ratio of the extinction coefficients of the major π-π* as well as the MLCT bands.

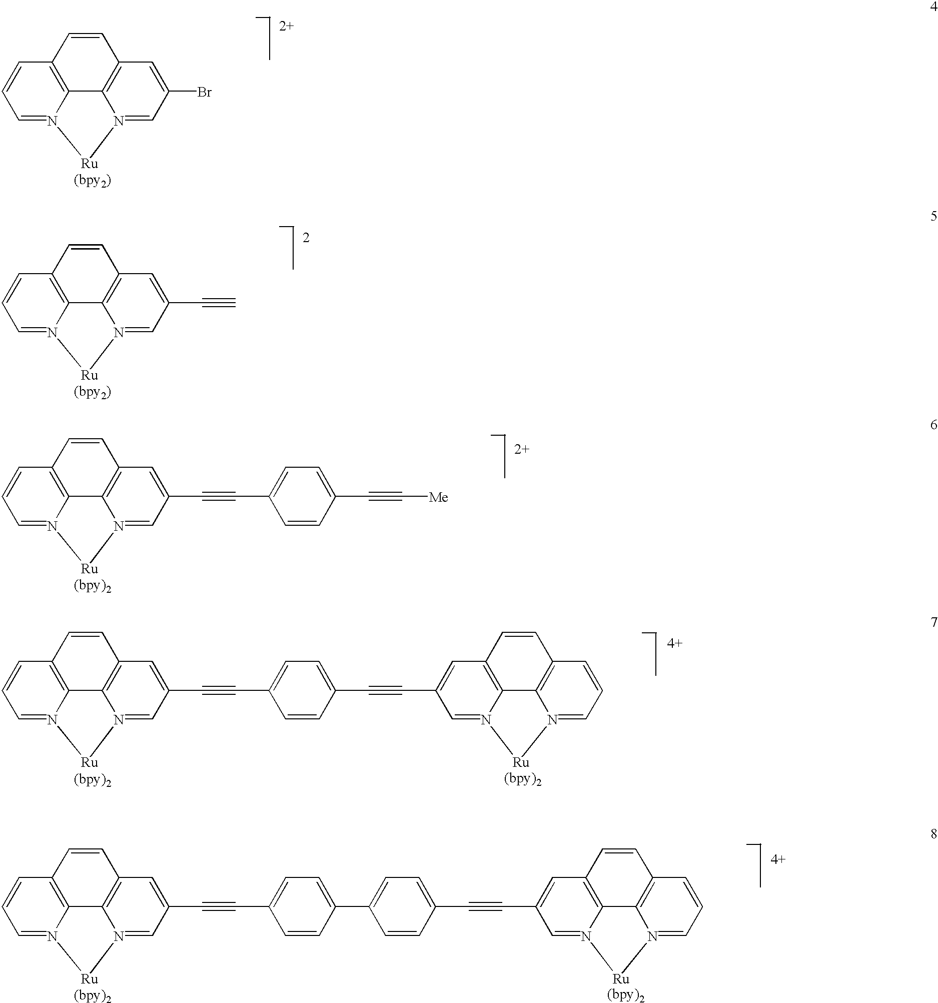

We claim:

1. A method for making acetylene derivatives of phenathrolines comprising reacting a 3,8 brominated phenanthroline with an aromatic acetylene.

2. The method of claim 1 wherein said 3,8 brominated phenathroline further comprises a transition metal atom.

3. The method of claim 1 wherein said 3,8 brominated phenathroline further comprises a chelated transition metal atom.

4. A compound having the formula comprising: 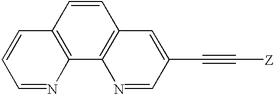
wherein Z is alkyl, substituted alkyl, aromatic or substituted aromatic group.

5. A compound according to claim 5 having the formula comprising 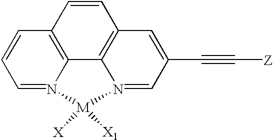
wherein
M is a transition metal ion; and
X and X₁ are co-ligands.

6. A compound according to claim 4 which is a polymer having the formula: 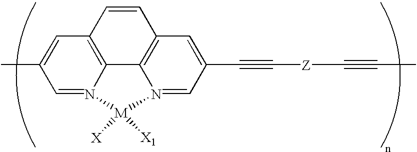
wherein
M is a transition metal ion and
X and X₁ are co-ligands.

7. A compound according to claim 6 having the formula:

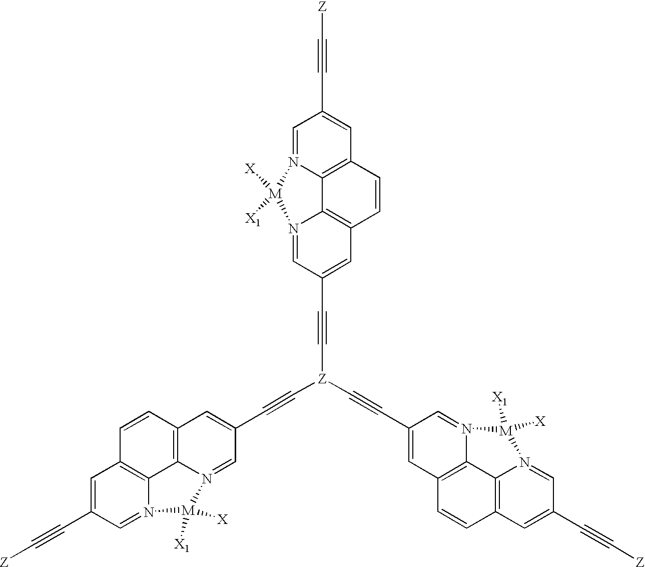

8. A compound according to claim 4 wherein Z comprises the base of a nucleoside.

9. A compound according to claim 4 wherein Z comprises the base of a nucleotide.

10. A compound according to claim 4 wherein Z comprises the phosphoramidite form of the nucleotide.
